SEQUENCE LISTING (24 sequences)

SEQ ID NO: 1 - DNA, 1386 bp, Oryza sativa
  source 1..1386: mol_type = genomic DNA
atggcgcgccgcgccgcttcccgcgctgttggctcggagggctcgatccaagggcgagggggccgcgcggggggcaatggcgccgaggacgcacgccacgtgttcgacgaattgcttcggcgtggcaagggcgccacgatctacggcttgaaccgcgccctcgacgacgtcgcgcgtcacagccccgcggccgccgtgtcccgctacaaccgcatggcccgagccggcgccgacgaggtaactcccaacttgtacacctacagcgttctcatcggttgctgctgccgggcgggccgcttggacctcggtttcgcggccttgggcaatgtcattaagaagggatttagagtggaagccatcaccttcactcctctgctcaagggcctctgtgccgacaagaggacgagcgacgcaatggacatagtgctctgcagaatgacccagctcggctgcataccaaatgtcttctcctgcaccattcttctcaagggtctgtgtgatgagaacagaagccaagaagctctcgagctgctccaaatgatgcctgatgatggaggtgactgcccacctgatgtggtgttgtacaacaccgtcatcaatggcttcttcaaagagggggatccggacaaagcttacgctacataccatgaaatgtttgaccaggggattttgccagatgttgtgacttacagctctattatcgctgccttatgcaaggctcaagctatggacaaggccatggaggtacttaacaccatggttaagaatggtgtcatgcctaattgcaggacatataatagtattgtgcacggatattgctcttcagggcagttgacagaggctattggatttctcaaaatgatgtgcagtgatggtgtcgaaccagatgttgttacttgtaacttgctgatggattatctttgcaagaacagaagatgcacggaagctagaaagattttcaattctatgaccaagtgtggcctaaagcctgatattactacctattgtaccctgcttcaggggtatgctaccaaaggagcccttgttgagatgcatgatctcctggatttgatggtatggaacggtatccaacctaatcatcatgtattcaacattctaatatgtgcatacgctaaacaagaaaaagtagatgaggcgatgcttgtattcagcaaaatgaggcagcaaggattgagtccgaatgcagtgaactacagaacagtcatagatgtactctgcaagctaggcagagtatacgatgcagtgcttaccttaaagcagatgatcaatgaaggactaacccctgacatcattgtatatacccccctaattcatggtttttgtacctgtgacaaatgggagaaggctgaggagttaattttttaa

SEQ ID NO: 2 - DNA, 945 bp, Oryza sativa
  source 1..945: mol_type = genomic DNA
atgatttctaaagccgaacgtcttttttgtcgactgtgtggtaaactagtttaccatgcggataagctttgttcgattatgcatgatgatccgataatcattctacttattcttctgattttggttttcgtatcacggatgttggttcgcgctttatggccccgttctgcagtattgacgtctcgcattttcactacttttgttgttctttcttcagcggttttgagtggatcggtggcacacgccgctgataacgaacctattaataatggggcagctcccgctgcttacggatccggggcagaaggaccctcaaattccgccttattcacttatacttccgatttggcggaagattcggtcagttccgggcgtagtagaagtaccgcctcgtcctcctttcaagaaagcacgggggaaatggacgcgctgatggcatcaacgactccttccgcgccgcggacaccctccgggggggaaccttcggtcaatcaaccgcttccaggggagcaagctatgcctcccgctcttcccgttatgcaggaagctgctaatcgggctctgccctacgcgccctacccgtatccagttgacgagataataggaggggattgcgtgcaatccattcaaaggagaattttgggagctaatttgaatccttccgcccatgacatgcaaatgtcccggattcaagcggaggatctatttgaactgaaagttcaaatcataagaaagatggcgaccctgcatccaagtggcgattggatgggatggggcgcgcgggccttggacaacccccgtacggccactggcgaggaagacttggctaagttgcacaaaatgctcgacgacctacagagccggaatgagcaatccgctaccttctggcgcttggtcgaaagagtccgcttacgggcggatgaggatcaaaactccgcttcctag

SEQ ID NO: 3 - protein, 314 aa, Oryza sativa
MISKAERLFCRLCGKLVYHADKLCSIMHDDPIIILLILLILVFVSRMLVRALWPRSAVLTSRIFTTFVVLSSAVLSGSVAHAADNEPINNGAAPAAYGSGAEGPSNSALFTYTSDLAEDSVSSGRSRSTASSSFQESTGEMDALMASTTPSAPRTPSGGEPSVNQPLPGEQAMPPALPVMQEAANRALPYAPYPYPVDEIIGGDCVQSIQRRILGANLNPSAHDMQMSRIQAEDLFELKVQIIRKMATLHPSGDWMGWGARALDNPRTATGEEDLAKLHKMLDDLQSRNEQSATFWRLVERVRLRADEDQNSAS

SEQ ID NO: 4 - DNA, 2349 bp, Oryza sativa
  source 1..2349: mol_type = genomic DNA
atggcgcgccgcgtccctacccgcccgcgcggcggtggcggcggcggcgtcccacgctcggagggctcgatccaagggcgaggaggccgcgcggggggcagtggcgccgaggacgcacgccacgtgttcgacgaattgctccggcgtggcaggggcgcctcgatctacggcttgaaccgcgccctcgccgacgtcgcgcgtcacagccccgcggccgccgtgtcccgctacaaccgcatggcccgagccggcgccggcaaggtaactcccaccgtgcacacctatgccatcctcatcggctgctgctgccgtgcgggccgcttggacctcggtttcgcggccttgggcaatgtcgtcaagaagggatttagagtggatgccatcaccttcactcctctgctcaagggcctctgtgccgacaagaggacgagcgacgcaatggacatagtgctccgcagaatgaccgagctcggctgcataccagatgtcttctcctacaataatcttctcaagggtctgtgtgatgagaacagaagccaagaagctctcgagctgctgcacatgatggctgatgatcgaggaggaggtagcccacctgatgtggtgtcgtataacactgtcctcaatggcttcttcaaagagggggattcagacaaagcttacagtacataccatgaaatgctggaccgggggattttaccagatgttgtgacctacagctctattattgctgcgttatgcaaggctcaagctatggacaaagccatggaggtacttaacaccatggttaagaatggtgtcatgcctgattgcatgacatataatagtattctgcatggatattgctcttcagggcagccaaaagaggctattggaacactcaaaaagatgcgcagtgatggcgtcgaaccaaatgttgttacttatagttcactgatgaattatctttgcaagaatggaagatccaccgaagctagaaagattttcgattctatgaccaagaggggcctagagcctgatattgctacctatcgtaccctgcttcaggggtatgctaccaaaggagcccttgttgagatgcatgctctcttggatttgatggtacgaaatggtatccaaccggatcatcatgtattcaacattctaatatgtgcatacgctaaacaagagaaagtagatcaggcaatgcttgtattcagcaaaatgaggcagcatggattgaatccgaatgtagtgtgctatggaacagttatagatgtactttgcaagtcaggcagtgtagatgatgctatgctttattttgagcagatgatcgatgaaggactaacccctaacattattgtgtatacctccctaattcatggtctgtgcacctgtgacaaatgggacaaggctgaagagttaattcttgaaatgttggatcgaggcatctgtctgaacactattttctttaattcaataattgacagtcattgcaaagaagggagggttatagaatctgaaaaactctttgacttgatggtacgaattggtgtgaagcccgatatcattacgtacaatacactcatcgatggatgctgcttagctggtaagatggatgaagcaacgaagttacttgccagcatggtctcagttggggtgaaacctgatattgttacctatggcaccttgattaatggctactgtagagttagcaggatggatgacgcattagctcttttcaaagagatggtgagcagtggtgttagtcctaatattattacgtataacataattctgcaaggtttatttcataccagaagaactgctgctgcaaaagaactctatgtcagtattaccaaaagtggaacacagcttgaacttagcacgtacaacataatccttcatggactttgcaaaaacaatctcactgacgaggcacttcgaatgtttcagaacctatgtttgacggatttacagctggagactaggacttttaacattatgattggtgccttacttaaatgtggaagaatggatgaagctaaggatttgtttgctgctcactcggctaacggtttagtgccagatgttaggacctacagtttaatggcagaaaatcttatagagcaggggtcgctagaagaattggatgatctatttctttcaatggaggagaatggctgttccgccgactcccgcatgctaaattccattgttaggaaactgttacagaggggtgatataaccagggctggcacttacctgttcatgattgatgagaagcacttctccctcgaagcatccactgcttccttcttgttagaatcttccccaatcgtctgggagcaaatatcaagaatatcgtag

SEQ ID NO: 5 - DNA, 2385 bp, Oryza sativa
  source 1..2385: mol_type = genomic DNA
atggcgcgccgcgccgcttcccgcgtccgcgccggcgctgttggcgcccttcgctcggagggctcgacccaagggcgagggggccgcacggggggcagtggcgccgaggacgcacgccacgtgttcgacgaattgctccggcgtggcaggggcgcctcgatctacggcttgaactgcgccctcgccgacgtcgcgcgtcacagccccgcggccgccgtgtcccgctacaaccgcatggcccgagccggcgccgacgaggtaactcccaacttgtgcacctacggcattctcatcggttcctgctgctgcgcgggccgcttggacctcggtttcgcggccttgggcaatgtcattaagaagggatttagagtggacgccatcgccttcactcctctgctcaagggcctctgtgctgacaagaggacgagcgacgcaatggacatagtgctccgcagaatgacccagcttggctgcataccaaatgtcttctcctacaatattcttctcaaggggctgtgtgatgagaacagaagccaagaagctctcgagctgctccaaatgatgcctgatgatggaggtgactgcccacctgatgtggtgtcgtataccactgtcatcaatggcttcttcaaggagggggatctggacaaagcttacggtacataccatgaaatgctggaccgggggattttaccaaatgttgttacctacagctctattattgctgcgttatgcaaggctcaagctatggacaaagccatggaggtacttaccagcatggttaagaatggtgtcatgcctaattgcaggacgtataatagtatcgtgcatgggtattgctcttcagggcagccgaaagaggctattggatttctcaaaaagatgcacagtgatggtgtcgaaccagatgttgttacttataactcgctcatggattatctttgcaagaacggaagatgcacggaagctagaaagatgttcgattctatgaccaagaggggcctaaagcctgaaattactacctatggtaccctgcttcaggggtatgctaccaaaggagcccttgttgagatgcatggtctcttggatttgatggtacgaaacggtatccaccctaatcattatgttttcagcattctaatatgtgcatacgctaaacaagggaaagtagatcaggcaatgcttgtgttcagcaaaatgaggcagcaaggattgaatccggatacagtgacctatggaacagttataggcatactttgcaagtcaggcagagtagaagatgctatgcgttattttgagcagatgatcgatgaaagactaagccctggcaacattgtttataactccctaattcatagtctctgtatctttgacaaatgggacaaggctaaagagttaattcttgaaatgttggatcgaggcatctgtctggacactattttctttaattcaataattgacagtcattgcaaagaagggagggttatagaatctgaaaaactctttgacctgatggtacgtattggtgtgaagcccgatatcattacgtacagtactctcatcgatggatattgcttggcaggtaagatggatgaagcaacgaagttacttgccagcatggtctcagttggaatgaaacctgattgtgttacatataatactttgattaatggctactgtaaaattagcaggatggaagatgcgttagttctttttagggagatggagagcagtggtgttagtcctgatattattacgtataatataattctgcaaggtttatttcaaaccagaagaactgctgctgcaaaagaactctatgtcgggattaccgaaagtggaacgcagcttgaacttagcacatacaacataatccttcatgggctttgcaaaaacaatctcactgacgaggcacttcgaatgtttcagaacctatgtttgacggatttacagctggagactaggacttttaacattatgattggtgcattgcttaaagttggcagaaatgatgaagccaaggatttgtttgcagctctctcggctaacggtttagtgccagatgttaggacctacagtttaatggcagaaaatcttatagagcaggggttgctagaagaattggatgatctatttctttcaatggaggagaatggctgtactgccaactcccgcatgctaaattccattgttaggaaactgttacagaggggtgatataaccagggctggcacttacctgttcatgattgatgagaagcacttctccctcgaagcatccactgcttccttgtttttagatcttttgtctgggggaaaatatcaagaatatcataggtttctccctgaaaaatataagtcctttatagaatctttgagctgctga

SEQ ID NO: 6 - protein, 461 aa, Oryza sativa
MARRAASRAVGSEGSIQGRGGRAGGNGAEDARHVFDELLRRGKGATIYGLNRALDDVARHSPAAAVSRYNRMARAGADEVTPNLYTYSVLIGCCCRAGRLDLGFAALGNVIKKGFRVEAITFTPLLKGLCADKRTSDAMDIVLCRMTQLGCIPNVFSCTILLKGLCDENRSQEALELLQMMPDDGGDCPPDVVLYNTVINGFFKEGDPDKAYATYHEMFDQGILPDVVTYSSIIAALCKAQAMDKAMEVLNTMVKNGVMPNCRTYNSIVHGYCSSGQLTEAIGFLKMMCSDGVEPDVVTCNLLMDYLCKNRRCTEARKIFNSMTKCGLKPDITTYCTLLQGYATKGALVEMHDLLDLMVWNGIQPNHHVFNILICAYAKQEKVDEAMLVFSKMRQQGLSPNAVNYRTVIDVLCKLGRVYDAVLTLKQMINEGLTPDIIVYTPLIHGFCTCDKWEKAEELIF

SEQ ID NO: 7 - protein, 782 aa, Oryza sativa
MARRVPTRPRGGGGGGVPRSEGSIQGRGGRAGGSGAEDARHVFDELLRRGRGASIYGLNRALADVARHSPAAAVSRYNRMARAGAGKVTPTVHTYAILIGCCCRAGRLDLGFAALGNVVKKGFRVDAITFTPLLKGLCADKRTSDAMDIVLRRMTELGCIPDVFSYNNLLKGLCDENRSQEALELLHMMADDRGGGSPPDVVSYNTVLNGFFKEGDSDKAYSTYHEMLDRGILPDVVTYSSIIAALCKAQAMDKAMEVLNTMVKNGVMPDCMTYNSILHGYCSSGQPKEAIGTLKKMRSDGVEPNVVTYSSLMNYLCKNGRSTEARKIFDSMTKRGLEPDIATYRTLLQGYATKGALVEMHALLDLMVRNGIQPDHHVFNILICAYAKQEKVDQAMLVFSKMRQHGLNPNVVCYGTVIDVLCKSGSVDDAMLYFEQMIDEGLTPNIIVYTSLIHGLCTCDKWDKAEELILEMLDRGICLNTIFFNSIIDSHCKEGRVIESEKLFDLMVRIGVKPDIITYNTLIDGCCLAGKMDEATKLLASMVSVGVKPDIVTYGTLINGYCRVSRMDDALALFKEMVSSGVSPNIITYNIILQGLFHTRRTAAAKELYVSITKSGTQLELSTYNIILHGLCKNNLTDEALRMFQNLCLTDLQLETRTFNIMIGALLKCGRMDEAKDLFAAHSANGLVPDVRTYSLMAENLIEQGSLEELDDLFLSMEENGCSADSRMLNSIVRKLLQRGDITRAGTYLFMIDEKHFSLEASTASFLLESSPIVWEQISRIS

SEQ ID NO: 8 - protein, 794 aa, Oryza sativa
MARRAASRVRAGAVGALRSEGSTQGRGGRTGGSGAEDARHVFDELLRRGRGASIYGLNCALADVARHSPAAAVSRYNRMARAGADEVTPNLCTYGILIGSCCCAGRLDLGFAALGNVIKKGFRVDAIAFTPLLKGLCADKRTSDAMDIVLRRMTQLGCIPNVFSYNILLKGLCDENRSQEALELLQMMPDDGGDCPPDVVSYTTVINGFFKEGDLDKAYGTYHEMLDRGILPNVVTYSSIIAALCKAQAMDKAMEVLTSMVKNGVMPNCRTYNSIVHGYCSSGQPKEAIGFLKKMHSDGVEPDVVTYNSLMDYLCKNGRCTEARKMFDSMTKRGLKPEITTYGTLLQGYATKGALVEMHGLLDLMVRNGIHPNHYVFSILICAYAKQGKVDQAMLVFSKMRQQGLNPDTVTYGTVIGILCKSGRVEDAMRYFEQMIDERLSPGNIVYNSLIHSLCIFDKWDKAKELILEMLDRGICLDTIFFNSIIDSHCKEGRVIESEKLFDLMVRIGVKPDIITYSTLIDGYCLAGKMDEATKLLASMVSVGMKPDCVTYNTLINGYCKISRMEDALVLFREMESSGVSPDIITYNIILQGLFQTRRTAAAKELYVGITESGTQLELSTYNIILHGLCKNNLTDEALRMFQNLCLTDLQLETRTFNIMIGALLKVGRNDEAKDLFAALSANGLVPDVRTYSLMAENLIEQGLLEELDDLFLSMEENGCTANSRMLNSIVRKLLQRGDITRAGTYLFMIDEKHFSLEASTASLFLDLLSGGKYQEYHRFLPEKYKSFIESLSC

SEQ ID NO: 9 - DNA, 20 bp, synthetic construct
  source 1..20: mol_type = other DNA
gaccacttcgttcgattcct

SEQ ID NO: 10 - DNA, 19 bp, synthetic construct
  source 1..19: mol_type = other DNA
atgcgacagccaaacaaac

SEQ ID NO: 11 - DNA, 807 bp, Lactuca sativa
  source 1..807: mol_type = genomic DNA
atgcccgcattaagatttaaaacttgtcgtctacttgaaggaaatgtttggaacaggaaacttacaataatacaacgccgcattcttcgaagattgaggaacaagacgagatctattaagagaatgatttattctcgaaaaaatctgaatagttacatccaattacaaactacacgaaagttgccccttgatttgaattttatctttgttatcagtacctttttttttttcattagctttggcgttccggttgcctttagcgccggcgagagtctttcagccgtaccttcccattcggattggtttacctttaccgaggatatgctggaagactcagcgagttccgggcgtagtagtagtacctcagcggtaaatcaaccgcttccgggggaacaagctacgcctcccgctcttccgggagctcggactccgccctacaccccctacccctatggcccggaggaagtgataggaggggattcggtccgctctatagagggccgcctcctgtcgggcaaaacagcgccttcgtcgctagatatatatctagcccggatcaacgccgaggacctattcgaggtcaaggtggatatcatccgggagatggcgggcctgcatccaagtggcgattggctggggcggggggctcgagccctcgataatccgagggggaaggagtccttgggggagctccttcaaatgcgtagagacctacgggctaggaatttgaaatccgagaccttcggcctattaaaggagaaggttttccttagggatacccccccctcagaaggctcttccgcatag

SEQ ID NO: 12 - DNA, 816 bp, Glycine max
  source 1..816: mol_type = genomic DNA
atgacgactcgcgcttttttcattctctttttttttcggtatgccgctccgccagcaaggagcgaaagaaccaagtggtttgtggtaatgtcagaatttgcacctatttgtctctcattcgtgatcagtctgctagtttctttgatcttcctcggtcttctttttgttaatccatcacaaactacacgaaagttgcccccttattttctctttgtttttcttatggctctcgcctgtagcattccgattgccttttgtgccagggccgacgacggtgccgtaccttccccttcggatttattaacgtatacatccgacatgctcgaagactcggcaagttccgggcgtagtagtagtacctcagcggtcaatcaaccgcttccgggggaacaagctatgcctcccgctcttcctgttatgcaggaagctgctaatcgggctctgccctacccctatcaaaataatgagattatagggggggacagtgttgaatccatccaacggaggcttttggggagattcccctctccttcagcccatgagattcaaatggcccggattgaagccgaagacctattcgaggtcaaggtcgatatttgtcaggtcatggcgggccttcacccaagcggggattggatgggacggggggctagagctctggataatcttcgtaccgccactggagaggagtccttaagtaagctccttcgaatgcgggaggacctacagactgcgggtttgcagtccgcaaccttccggcaattggccgacagagtggcattccgggcggatggagatcaacactctgccacctag

SEQ ID NO: 13 - DNA, 915 bp, Solanum lycopersicum
  source 1..915: mol_type = genomic DNA
atgtcaccttttttctttagaggcgttctctctcttattgcaacctttctggggcatttcgctgttagagtaatgcccattcgaataactggtaagtgtctcatgctgttccttttagtgattatggggttgatccttattttgagatttttgggttttcgttcaaagagaaaataccggtttattaatctatttattttgattcttattttatttagtatctctttatttatattttttctaaggaactctttgctatcccatttagggattttgtgttctcagcttctctctgggattttgctttttagtgtcgggggatcacttcctcttccgggcccttccgggcaagcaagcggggctgagtttgaagaggatcattttgggattgatgtcctcttggaatcatgggaaaaatctacggaaacgggcacgtcggtggatcagccagaaccagaaccaggacatgtgccgcctgctattcaagtagctccccgggggaacgaagctggtccatcgaatcagcccccacgagtggtcccctatccgtatcaactggatgaagtgatagggggggattccgttcagtctatccagcggaggctcctagccaaatactcctatactccccctatcgggatcatgtacctggccgaaatagaggcaaaagacctattcgaggtcaaggtcgagattctaaggatcatggcggtccttgatccaacgggggattggctgggacggggagctcgggccctcgagaatccgcgtaccgccacgggagagcattccttggataaactctatacccttctttcggatctcgaatcgaggggagtcaattccgagtccttctctcaattaaaagggaaggtacccctgcgaaggggttgggacgaacactctaccacatag

SEQ ID NO: 14 - protein, 268 aa, Lactuca sativa
MPALRFKTCRLLEGNVWNRKLTIIQRRILRRLRNKTRSIKRMIYSRKNLNSYIQLQTTRKLPLDLNFIFVISTFFFFISFGVPVAFSAGESLSAVPSHSDWFTFTEDMLEDSASSGRSSSTSAVNQPLPGEQATPPALPGARTPPYTPYPYGPEEVIGGDSVRSIEGRLLSGKTAPSSLDIYLARINAEDLFEVKVDIIREMAGLHPSGDWLGRGARALDNPRGKESLGELLQMRRDLRARNLKSETFGLLKEKVFLRDTPPSEGSSA

SEQ ID NO: 15 - protein, 271 aa, Glycine max
MTTRAFFILFFFRYAAPPARSERTKWFVVMSEFAPICLSFVISLLVSLIFLGLLFVNPSQTTRKLPPYFLFVFLMALACSIPIAFCARADDGAVPSPSDLLTYTSDMLEDSASSGRSSSTSAVNQPLPGEQAMPPALPVMQEAANRALPYPYQNNEIIGGDSVESIQRRLLGRFPSPSAHEIQMARIEAEDLFEVKVDICQVMAGLHPSGDWMGRGARALDNLRTATGEESLSKLLRMREDLQTAGLQSATFRQLADRVAFRADGDQHSAT

SEQ ID NO: 16 - protein, 304 aa, Solanum lycopersicum
MSPFFFRGVLSLIATFLGHFAVRVMPIRITGKCLMLFLLVIMGLILILRFLGFRSKRKYRFINLFILILILFSISLFIFFLRNSLLSHLGILCSQLLSGILLFSVGGSLPLPGPSGQASGAEFEEDHFGIDVLLESWEKSTETGTSVDQPEPEPGHVPPAIQVAPRGNEAGPSNQPPRVVPYPYQLDEVIGGDSVQSIQRRLLAKYSYTPPIGIMYLAEIEAKDLFEVKVEILRIMAVLDPTGDWLGRGARALENPRTATGEHSLDKLYTLLSDLESRGVNSESFSQLKGKVPLRRGWDEHSTT

SEQ ID NO: 17 - DNA, 23 bp, synthetic construct
  source 1..23: mol_type = other DNA
tacacctacagcgttctcatcgg

SEQ ID NO: 18 - DNA, 23 bp, synthetic construct
  source 1..23: mol_type = other DNA
tcgtataacactgtcctcaatgg

SEQ ID NO: 19 - DNA, 23 bp, synthetic construct
  source 1..23: mol_type = other DNA
atcggttcctgctgctgcgcggg

SEQ ID NO: 20 - DNA, 22 bp, synthetic construct
  source 1..22: mol_type = other DNA
ttatgcaaggctcaagctatgg

SEQ ID NO: 21 - DNA, 20 bp, synthetic construct
  source 1..20: mol_type = other DNA
tacgaacaaccgaggatgag

SEQ ID NO: 22 - DNA, 20 bp, synthetic construct
  source 1..20: mol_type = other DNA
gattcccccttctctcagcc

SEQ ID NO: 23 - DNA, 20 bp, synthetic construct
  source 1..20: mol_type = other DNA
gagagcctgacctattgcat

SEQ ID NO: 24 - DNA, 20 bp, synthetic construct
  source 1..20: mol_type = other DNA
tcggcgagtacttctacaca

(19) United States
(12) Patent Application Publication
(10) Pub. No.: US 2026/0258440 A1
(43) Pub. Date: Sep. 3, 2026

(54) METHOD FOR PRODUCING CYTOPLASMIC MALE STERILE LINE AND MAINTAINER LINE

(71) Applicant: TOHOKU UNIVERSITY, Miyagi (JP)

(72) Inventors: Kinya TORIYAMA, Miyagi (JP); Keisuke IGARASHI, Miyagi (JP); Yuko IWAI, Miyagi (JP); Shinya TAKEDA, Miyagi (JP); Ayumu TAKATSUKA, Miyagi (JP); Tomohiko KAZAMA, Miyagi (JP)

(73) Assignee: TOHOKU UNIVERSITY, Miyagi (JP)

(21) Appl. No.: 19/163,178

(22) PCT Filed: Mar. 8, 2024
(86) PCT No.: PCT/JP2024/009165

§ 371 Date: Sep. 8, 2025

(30) Foreign Application Priority Data
Mar. 9, 2023 (JP) 2023-036941

(51) Int. Cl. C12N 15/82

(52) U.S. Cl. CPC C12N 15/8289

(57) ABSTRACT

An object to be achieved by the present invention is to provide a novel system that can produce a cytoplasmic male sterile line and a maintainer line from one and the same species. The present invention provides a method of producing a cytoplasmic male sterile line plant, including a step of knocking out, in a plant having an orf288 gene in mitochondrial genomic DNA thereof and having a pentatricopeptide repeat protein (PPR) gene in nuclear genomic DNA thereof, the PPR gene in the nuclear genomic DNA.

### TECHNICAL FIELD

The present invention relates to methods of producing a cytoplasmic male sterile line and a maintainer line.

### BACKGROUND ART

An F₁ hybrid breeding method is also referred to as “hybrid variety breeding method,” and has been utilized in breeding because a variety having both the excellent traits of its parents and showing heterosis can be bred. To grow a large amount of F₁ seeds at low cost, a three-line method utilizing cytoplasmic male sterility has been utilized in the production of F₁ hybrid seeds. The term “three-line method” refers to a method utilizing: a cytoplasmic male sterile line serving as a line carrying a male sterile cytoplasm; a fertility restorer line carrying, in its nucleus, a restorer-of-fertility gene corresponding to the male sterile cytoplasm; and a maintainer line serving as a line, whose nuclear gene is identical to that of the sterile line and which does not carry any sterile cytoplasm. Through use of those three lines, (i) the F₁ hybrid seeds can be obtained by crossing the sterile line with the pollen of the restorer line, and (ii) meanwhile, the sterile line can be maintained and proliferated by crossing the sterile line with the pollen of the maintainer line.

The production of the F₁ hybrid seeds through use of the cytoplasmic male sterility has been put into practical use in, for example, a corn, sorghum, a sugar beet, a sunflower, cruciferous vegetables, such as a broccoli and a Japanese white radish, a carrot, celery, a leek, and an onion, and has been widely used.

### CITATION LIST

### Patent Literature

PTL 1: JP 2018-130043 A

### Non-Patent Literature

NPL 1: Nat Genet 45: 573-577 (2013)
NPL 2: Rice 2014, 7:28: 1-5 (2014)
NPL 3: Mol Plant 7: 1497-1500 (2014)
NPL 4: Cell Res 27: 130-146 (2017)
NPL 5: Nucleic Acids Research 47: 3728-3738 (2019)
NPL 6: Plant Mol Biol 88: 561-572 (2015)
NPL 7: Proc Natl Acad Sci USA 108: 1723-1728 (2011)
NPL 8: Breeding Research 20 (Supplement 1): 121 (2018)
NPL 9: Breeding Research 23 (Supplement 2): 108 (2021)

### SUMMARY OF INVENTION

### Technical Problem

Many male sterile cytoplasms are derived from, for example, a wild relative and a subspecies that is identical in kind to the cytoplasms but is distantly related thereto, and hence a cytoplasmic male sterile line has been bred by successively backcrossing the wild relative or the distant subspecies with a cultivar (FIG. 1). However, the wild relative and the distant subspecies that can be utilized are limited, and hence the development of a novel system that can produce the cytoplasmic male sterile line from an existing variety alone has been demanded from the seed industry.

### Solution to Problem

Cytoplasmic male sterility (CMS) is caused by the expression of a CMS-causing gene present in the mitochondria of a wild relative or a distant subspecies. None of the wild relative and the distant subspecies shows male sterility because each of the wild relative and the distant subspecies carries a restorer-of-fertility gene in its nucleus to suppress the expression of the CMS-causing gene present in its mitochondria. Meanwhile, when the nucleus is substituted with the nucleus of a cultivar free of any restorer-of-fertility gene, the restorer-of-fertility gene is lost, and hence the CMS-causing gene may be expressed to make the wild relative or the distant subspecies cytoplasmic male sterile.

For example, a WA-type cytoplasmic male sterile line derived from Hainan Island wild rice causes male sterility as follows (Non-patent Literature 1): a CMS-causing gene WA352 is present in its mitochondrial genome, and a WA352 protein accumulates in a meiotic-stage anther to cause the sterility. The WA352 forms the chimeric structures of genes orf284, orf224, and orf288 having unknown functions, which are present in the mitochondrial genome of a rice variety “Nipponbare”. It has been known that the WA352 protein causes male sterility because an ORF288 homologous sequence binds to the subunit COX11 of the cytochrome C oxidase complex of the electron transport system of its mitochondria to produce reactive oxygen species (Non-patent Literature 4). The orf288 gene of the Nipponbare encodes 314 amino acids. It has been reported that although no orf288 RNA can be detected in the anther of the Nipponbare, when an orf288 protein is expressed in yeast, the protein binds to the COX11 as in the WA352 protein (Non-patent Literature 4).

Meanwhile, an Rf4 gene is cloned as a restorer-of-fertility gene. The Rf4 encodes a pentatricopeptide repeat protein serving as a sequence-specific RNA-binding protein (Non-patent Literature 2 and Non-patent Literature 3). The presence of the Rf4 degrades WA352 RNA, and hence the male sterility of the WA-type cytoplasmic male sterile line is avoided.

It has been reported that when African rice Oryza glaberrima is successively backcrossed by using a cultivated rice variety “Taichung 65” as a mother, a cytoplasmic male sterile line is obtained (Non-patent Literature 8). In the cytoplasmic male sterile line, mitochondria are the “Taichung 65,” and a nucleus is the O. glaberrima. The foregoing means that the cultivated rice variety “Taichung 65” conceals a CMS-causing gene in its mitochondrial genome. Meanwhile, with regard to a fertility restorer line, it has been reported that although mitochondria are the “Taichung 65,” and a nucleus is the O. glaberrima, 1.5 Mb of a fragment derived from the “Taichung 65” remains in a tenth chromosome, and at least two restorer-of-fertility genes that can control the expression of a CMS gene board thereon in sequence (Non-patent Literature 8). When the expression of the orf288 gene is investigated in each of the meiotic-stage anthers of the CMS line (TGA) and the fertility restorer line (TGR), orf288 RNA can be detected in the CMS line, but substantially no orf288 RNA can be detected in the fertility restorer line. Accordingly, the orf288 gene has been conceived as a candidate for a potential CMS-inducing gene (Non-patent Literature 9).

Under such circumstances, the inventors of the present invention have made extensive investigations, and as a result, have predicted that the RNA of the orf288 is subjected to processing by a restorer-of-fertility gene, and hence the restorer-of-fertility gene is a PPR gene serving as a sequence-specific RNA-binding protein. The inventors of the present invention have used a trial and error approach to a large extent on the basis of such prediction, and as a result, have found that even when, in a plant having the orf288 gene in its mitochondrial genome and having the PPR gene in the nuclear genome, the PPR gene in the nuclear genome is knocked out, an influence on the growth of the plant is small, and a cytoplasmic male sterile line plant can be produced by releasing the suppression of the expression of the orf288 gene present in the mitochondrial genome. The inventors of the present invention have further found the following: when, in the plant having the orf288 gene in its mitochondrial genome and having the PPR gene in its nuclear genome, the PPR gene in the nuclear genome is knocked out, and the orf288 gene in the mitochondrial genome is further knocked out, the orf288 gene is not expressed, and hence pollen normally grows; however, the gene of the mitochondrial genome is transported only through a seed parent (maternal gene), and hence a maintainer line that can produce a cytoplasmic male sterile line is obtained by crossing the cytoplasmic male sterile line with the line in which both the PPR gene and the orf288 gene have been knocked out (FIG. 1). The present invention is based on such novel findings. The inventors of the present invention have used a trial and error approach to a large extent on the basis of the above-mentioned findings, and have finally completed the present invention. Accordingly, the present invention provides the following items.

Item 1. A method of producing a cytoplasmic male sterile line plant, including a step of knocking out, in a plant having an orf288 gene in mitochondrial genomic DNA thereof and having a pentatricopeptide repeat protein (PPR) gene in nuclear genomic DNA thereof, the PPR gene in the nuclear genomic DNA.

Item 2. A method of producing a maintainer line plant, including the steps of:
knocking out, in a plant having an orf288 gene in mitochondrial genomic DNA thereof and having a PPR gene in nuclear genomic DNA thereof, the PPR gene in the nuclear genomic DNA; and knocking out the orf288 gene in the mitochondrial genomic DNA.

Item 3. The method according to item 1 or 2, wherein the PPR gene satisfies all of the following requirements:
(1) the PPR gene is expressed in an anther of the plant;
(2) the PPR gene has a mitochondrial targeting signal; and
(3) the PPR gene has 10 or more PPR motifs.

Item 4. The method according to item 2, wherein the orf288 gene includes a base sequence encoding an amino acid sequence as set forth in SEQ ID NO: 3 or a base sequence encoding an amino acid sequence having a similarity of 80% or more to the amino acid sequence as set forth in SEQ ID NO: 3 in blast analysis.

Item 5. A combination of plants of three lines, including:
a restorer line plant, which is a plant having an orf288 gene in mitochondrial genomic DNA thereof and having a pentatricopeptide repeat protein (PPR) gene in nuclear genomic DNA thereof;
a cytoplasmic male sterile line plant obtained by knocking out, in the restorer line plant, the PPR gene in the nuclear genomic DNA; and
a maintainer line plant obtained by knocking out, in the restorer line plant, the PPR gene in the nuclear genomic DNA and the orf288 gene in the mitochondrial genomic DNA.

Item 6. An F₁ hybrid plant, which is produced through use of the combination of plants of three lines of item 5 and a combination of plants of three lines of other varieties by a three-line method.

Item 7. The combination according to item 5 or the plant according to item 6, wherein the PPR gene satisfies all of the following requirements:
(1) the PPR gene is expressed in an anther of the plant;
(2) the PPR gene has a mitochondrial targeting signal; and
(3) the PPR gene has 10 or more PPR motifs.

Item 8. The combination according to item 5 or the plant according to item 6, wherein the orf288 gene includes a base sequence encoding an amino acid sequence as set forth in SEQ ID NO: 3 or a base sequence encoding an amino acid sequence having a similarity of 80% or more to the amino acid sequence as set forth in SEQ ID NO: 3 in blast analysis.

### Advantageous Effects of Invention

According to the present invention, there can be provided the novel system that can produce a cytoplasmic male sterile line from one and the same species. In addition, according to the present invention, there can be provided the novel system that can produce a maintainer line from one and the same species.

### BRIEF DESCRIPTION OF DRAWINGS

FIG. 1 is a diagram for illustrating the outlines of a method of the present invention and a related-art method.

FIG. 2 is a photograph showing the results of a genotyping using a DNA marker SSRH10045 in a plant formed of Nipponbare and O. glaberrima (BC3F1). SSRH10045F GACCACTTCGTTCGATTCCT is as set forth in SEQ ID NO: 9, and SSRH10045R ATGCGACAGCCAAACAAAC is as set forth in SEQ ID NO: 10.

FIG. 3 is a set of microphotographs of the pollen of the plant formed of the Nipponbare and the O. glaberrima (BC3F1) with seed setting rates, and microphotographs of the pollen of a plant formed of Taichung 65 and the O. glaberrima (BC5F6) with seed setting rates.

FIG. 4 is a set of photographs showing the results of the northern blot analysis of orf288 in the meiotic-stage glumous flower of the plant formed of the Nipponbare and the O. glaberrima (BC3F1).

FIG. 5 is a photograph showing the selection of a null segregant obtained by deleting the orf288 from the Taichung 65 (T65) through the introduction of mito-TALENs thereinto.

FIG. 6 is a set of photographs showing the manner in which a Taichung 65 (T65) individual from which the orf288 has been deleted sets a seed.

FIG. 7-1 is a diagram for illustrating comparison between the amino acid sequences of the orf288 homologous gene (SEQ ID NO: 14) of a lettuce (Ls) and rice (Os). Highlighted portions represent interaction regions I and II between the WA352 of WA-CMS rice and a COX11.

FIG. 7-2 is a diagram for illustrating comparison between the amino acid sequences of the orf288 homologous gene (SEQ ID NO: 15) of a soybean (Gm) and the rice (Os). Highlighted portions represent the interaction regions I and II between the WA352 of the WA-CMS rice and the COX11.

FIG. 7-3 is a diagram for illustrating comparison between the amino acid sequences of the orf288 homologous gene (SEQ ID NO: 16) of a tomato (Sl) and the rice (Os). Highlighted portions represent the interaction regions I and II between the WA352 of the WA-CMS rice and the COX11.

FIG. 8 is a photograph showing the expression analysis of the orf288 in a PPR knock-out individual (T65/PPR_KO). The term “TGA” refers to a CMS line, the term “TGR” refers to a fertility restorer line, and the term “T65” refers to the Taichung 65.

### DESCRIPTION OF EMBODIMENTS

In the present invention, the term “gene” encompasses not only a structural gene that specifies the primary structure of, for example, a protein, tRNA, or rRNA, but also a region on a nucleic acid having a specific controlling function, such as a promoter or an operator, unless otherwise stated or unless it is described that the term does not mean such region. Accordingly, in the present invention, the term “gene” refers to a regulatory region, a coding region, an exon, and an intron without any distinction unless otherwise stated. In addition, the term “structural gene” encompasses silent DNA obtained by subjecting an original DNA sequence to silent mutation. In addition, in the present invention, a nucleic acid molecule that interferes with gene expression such as siRNA is also encompassed in the term “gene”.

The term “nucleic acid” as used herein is identical in meaning to a nucleotide, an oligonucleotide, and a polynucleotide, and may refer to any one of DNA, RNA, and a DNA-RNA hybrid. In addition, such nucleic acid may be double-stranded or single-stranded, and when the term refers to a nucleic acid molecule having a certain sequence, the term comprehensively means a nucleic acid molecule (or a nucleotide, an oligonucleotide, or a polynucleotide) having a sequence complementary thereto unless otherwise stated. In addition, such nucleic acid molecule may be cyclic or linear, and may be any one of a synthetic nucleic acid and a living organism-derived nucleic acid.

### Method of Producing Cytoplasmic Male Sterile Line Plant

In one embodiment, the present invention provides a method of producing a cytoplasmic male sterile line plant, including a step of knocking out, in a plant having an orf288 gene in its mitochondrial genome and having a pentatricopeptide repeat protein (PPR) gene in its nuclear genome, the PPR gene in the nuclear genome.

In the present invention, the plant of interest is not particularly limited, and any plant may be adopted as long as the plant is a seed plant. There may be used, for example, plants belonging to gramineous plants (e.g., rice, wheat, corn, barley, rye, and sorghum) and cruciferous plants (e.g., the genus Alyssum, the genus Arabidopsis (e.g., Arabidopsis thaliana), the genus Armoracia (e.g., horseradish), the genus Aurinia, the genus Brassica (e.g., tatsoi, mustard, Japanese giant red mustard, rape, mizuna, kale, ornamental cabbage, cauliflower, cabbage, Brussels sprout, broccoli, Bok choy, nozawana, wild turnip, napa cabbage, komatsuna, and turnip), the genus Camelina, the genus Capsella, the genus Cardamine, the genus Coronopus, the genus Diplotaxis, the genus Draba, the genus Eruca (e.g., rocket), the genus Hesperis, the genus Hirschfeldia, the genus Iberis, the genus Ionopsidium, the genus Lepidium, the genus Lobularia, the genus Lunaria, the genus Malcolmia, the genus Matthiola, the genus Nasturtium, the genus Orychophragmus, the genus Raphanus (e.g., daikon radish and radish), the genus Rapistrum, the genus Rorippa, the genus Sisymbrium, the genus Thlaspi, and the genus Eutrema (e.g., wasabi)). Examples thereof may also include: solanaceous plants, such as a tomato, a potato, a bell pepper, a shishito pepper, and a petunia; asteraceous plants, such as a lettuce, a sunflower, and a dandelion; convolvulaceous plants, such as a morning glory and a sweet potato; araceous plants, such as konjac, taro, eddo, and yatsugashira; leguminous plants, such as a soybean, an adzuki bean, and a common bean; cucurbitaceous plants, such as a pumpkin, a cucumber, and a melon; and amaryllidaceous plants, such as an onion, a green onion, and garlic.

In the present invention, the term “orf288 gene” encompasses not only an orf288 gene in the mitochondrial genomic DNA of rice but also a corresponding gene in any other plant (a gene in a plant except the rice, the gene corresponding to the orf288 gene in the mitochondrial genomic DNA of the rice, is also referred to as “orf288 homologous gene”). The orf288 gene is specifically, for example, a nucleic acid molecule including: a base sequence encoding an amino acid sequence as set forth in SEQ ID NO: 3 (the amino acid sequence of the orf288 of the rice); or a base sequence encoding an amino acid sequence having a similarity of 80% or more to the amino acid sequence as set forth in SEQ ID NO: 3 in blast analysis. In addition, the orf288 gene is, for example, a gene having: a base sequence as set forth in SEQ ID NO: 2 (the base sequence of the orf288 of the rice); or a base sequence having a homology of 70% or more (preferably 80% or more, more preferably 85% or more, more preferably 90% or more, more preferably 95% or more, more preferably 97% or more, more preferably 99% or more) to the base sequence as set forth in SEQ ID NO: 2.

The PPR gene preferably satisfies at least one of the following requirements, and more preferably satisfies all the requirements:
(1) the PPR gene is expressed in the anther of the plant;
(2) the PPR gene has a mitochondrial targeting signal; and
(3) the PPR gene has 10 or more PPR motifs.

In the embodiment, the mitochondrial targeting signal is, for example, a region from the 1-position to 231-position of a base sequence represented by NCBI accession No. AB110016, a region from the 1-position to 210-position of a base sequence represented by NCBI accession No. AB900792, or a region from the 1-position to 231-position of a base sequence represented by NCBI accession No. LC131122. In addition, the mitochondrial targeting signal is, for example, a signal having a base sequence obtained by substituting, deleting, or adding one or several (e.g., two or three) bases for, from, or to one or several bases of each of the regions of the base sequences described above. In addition, the mitochondrial targeting signal is, for example, a signal having a base sequence having a homology of 70% or more (preferably 80% or more, more preferably 85% or more, more preferably 90% or more, more preferably 95% or more, more preferably 97% or more, more preferably 99% or more) to each of the base sequences in the regions described above.

In addition, in the embodiment, it has been known that one PPR motif of a PPR protein recognizes one nucleotide, and the sequence of binding RNA has been predicted from the amino acid sequence of the PPR motif (Non-patent Literature 5). It may become possible to estimate an Rf-like PPR encoding a PPR protein binding to the RNA of the orf288 gene from a plurality of Rf-like PPR genes through use of the prediction or in conformity with the prediction. The number of the PPR motifs in the PPR gene that are expected to bind to the RNA of the orf288 gene is preferably 10 or more, more preferably 15 or more, still more preferably 20 or more.

In addition, the PPR gene preferably belongs to the group of restorer-of-fertility genes when genes in nuclear genomic DNA are divided into clusters by using ClustalW. In addition, whether or not the PPR gene belongs to the group of the restorer-of-fertility genes can be judged with reference to the description of Non-patent Literature 7. In addition, the PPR gene is preferably a gene that is expected to have a high binding property between its protein and orf288 RNA. The binding property with the orf288 RNA can be predicted with reference to the description of Non-patent Literature 5. More specifically, from the viewpoint of the binding property with the orf288 RNA, 1,575 or more bases serving as PPR motifs are preferably predicted to be present by using software TPRpred (https://toolkit.tuebingen.mpg.de/tools/tprpred). In addition, examples of the PPR gene include the following sequences:
a base sequence as set forth in SEQ ID NO: 1 (the base sequence of PPR461);
a base sequence having a homology of 70% or more (preferably 80% or more, more preferably 85% or more, more preferably 90% or more, more preferably 95% or more, more preferably 97% or more, more preferably 99% or more) to the base sequence as set forth in SEQ ID NO: 1;
a base sequence encoding an amino acid sequence as set forth in SEQ ID NO: 6 (the amino acid sequence of the PPR461);
a base sequence encoding an amino acid sequence having a similarity of 80% or more to the amino acid sequence as set forth in SEQ ID NO: 6 in blast analysis;
a base sequence as set forth in SEQ ID NO: 4 (the base sequence of PPR782);
a base sequence having a homology of 70% or more (preferably 80% or more, more preferably 85% or more, more preferably 90% or more, more preferably 95% or more, more preferably 97% or more, more preferably 99% or more) to the base sequence as set forth in SEQ ID NO: 4;
a base sequence encoding an amino acid sequence as set forth in SEQ ID NO: 7 (the amino acid sequence of the PPR782);
a base sequence encoding an amino acid sequence having a similarity of 80% or more to the amino acid sequence as set forth in SEQ ID NO: 7 in blast analysis;
a base sequence as set forth in SEQ ID NO: 5 (the base sequence of PPR794);
a base sequence having a homology of 70% or more (preferably 80% or more, more preferably 85% or more, more preferably 90% or more, more preferably 95% or more, more preferably 97% or more, more preferably 99% or more) to the base sequence as set forth in SEQ ID NO: 5;
a base sequence encoding an amino acid sequence as set forth in SEQ ID NO: 8 (the amino acid sequence of the PPR782); and
a base sequence encoding an amino acid sequence having a similarity of 80% or more to the amino acid sequence as set forth in SEQ ID NO: 8 in blast analysis.

In this embodiment, the method of the present invention includes the step of knocking out the PPR gene in the nuclear genome. A method of knocking out the PPR gene is not particularly limited, and the gene may be appropriately knocked out by using a known genome editing technology or the like. For example, the PPR gene may be disrupted by removing part (e.g., 10% or more, 20% or more, 30% or more, 40% or more, 50% or more, 60% or more, 70% or more, 80% or more, or 90% or more) or the entirety of the region of the PPR gene to be targeted through use of a genome-editing tool CRISPR-Cas9, or inserting a nucleic acid molecule into the region of the PPR gene.

In a restorer line plant, the expression of the orf288 gene in its mitochondrial genomic DNA is suppressed by the PPR gene. In this embodiment, however, the PPR gene is disrupted, and hence a suppressing effect on the expression of the orf288 gene no longer works or reduces while the plant grows. Accordingly, the orf288 gene is expressed to cause male sterility. Accordingly, the cytoplasmic male sterile line plant can be produced by the method. In this embodiment, it is important that the orf288 gene be caused to function, and hence the gene be not knocked out because the male sterility is caused by the expression of the orf288 gene.

### Method of Producing Maintainer Line Plant

In addition, in another embodiment, the present invention provides a method of producing a maintainer line plant, including the steps of: knocking out, in a plant having an orf288 gene in its mitochondrial genomic DNA and having a PPR gene in its nuclear genomic DNA, the PPR gene in the nuclear genomic DNA; and knocking out the orf288 gene in the mitochondrial genomic DNA.

The plant of interest, a method of knocking out the PPR gene, and the like are the same as those described in the section “Method of producing Cytoplasmic Male Sterile Line Plant.” In the embodiment of the method of producing a maintainer line plant, the method of the present invention includes not only the step of knocking out the PPR gene in the nuclear genomic DNA but also the step of knocking out the orf288 gene in the mitochondrial genomic DNA. A method of knocking out the orf288 gene in the mitochondrial genomic DNA is not particularly limited, and the gene may be knocked out by using a known method or in conformity with the known method. For example, the inventors of the present invention have developed a method of inducing a change in structure of the mitochondrial genome of a plant cell through the introduction of a double-strand break into a target sequence region present in each molecular species of mitochondrial genomic DNA in the plant cell (Patent Literature 1), and have obtained a patent therefor. Accordingly, even in the present invention, the orf288 gene in the mitochondrial genomic DNA may be knocked out by using the method described in Patent Literature 1. More specifically, to knock out the orf288 gene in the mitochondrial genomic DNA, methods described in the following items (1) to (8) may each be used while the orf288 gene is defined as a target sequence region:
(1) a method of inducing a change in structure of the mitochondrial genome of a plant cell through the introduction of a double-strand break into a target sequence region present in each molecular species of mitochondrial genomic DNA in the plant cell;
(2) the method according to the above-mentioned item (1), characterized in that the change in structure of the mitochondrial genome is induced by DNA recombination occurring between a sequence present near the target sequence region and a homologous sequence present in another region;
(3) a method of deleting a gene present in each molecular species of mitochondrial genomic DNA in a plant cell through the introduction of a double-strand break into the gene or a region near the gene;
(4) the method according to the above-mentioned item (3), characterized in that the deletion of the gene is induced by DNA recombination occurring between a sequence present in the gene or the region near the gene and a homologous sequence present in another region;
(5) the method according to the above-mentioned item (3) or (4), characterized in that the gene is a causative gene for male sterility;
(6) the method according to any one of the above-mentioned items (1) to (5), characterized in that the double-strand break is introduced by transcription activator-like effector nucleases (TALENs);
(7) the method according to the above-mentioned item (6), characterized in that a mitochondrial localization signal peptide derived from a protein localized in plant mitochondria is used for transferring the TALENs to mitochondria;
(8) the method according to the above-mentioned item (7), characterized in that the expression construct of the TALENs is adjusted by a method including the following steps (a) to (c):
(a) a step of providing an entry vector 1 in which a TALEN left is inserted between two homologous recombination sequences L1 and L4, an entry vector 2 in which a terminator, a promoter, and a mitochondrial localization signal are inserted in the stated order between two homologous recombination sequences R4 and R3, and an entry vector 3 in which a TALEN right is inserted between two homologous recombination sequences L3 and L2;
(b) a step of providing a destination vector into which the promoter, the mitochondrial localization signal, a homologous recombination sequence R1, and a homologous recombination sequence R2 have been inserted in the stated order; and
(c) a step of mixing the entry vector 1, the entry vector 2, the entry vector 3, and the destination vector to perform homologous recombination between the L1 and the R1, between the L4 and the R4, between the L3 and the R3, and between the L2 and the R2;
(9) the method according to any one of the items (1) to (8), characterized in that the induction of the change in structure of the mitochondrial genome is performed without occurrence of restoration by a heterologous terminal bond; and
(10) the method according to any one of the items (1) to (8), characterized in that the induction of the change in structure of the mitochondrial genome is the induction of the deletion/disappearance of a partial region of the mitochondrial genomic DNA and the reconstruction of a new mitochondrial genomic DNA sequence.

According to the method of the present invention in this embodiment, in the plant having the orf288 gene in the mitochondrial genomic DNA and having the PPR gene in the nuclear genomic DNA, the PPR gene in the nuclear genomic DNA and the orf288 gene in the mitochondrial genomic DNA are knocked out. Thus, while the plant grows, a suppressing effect exhibited by the PPR gene on the expression of the orf288 gene no longer works. Meanwhile, the orf288 gene itself is disrupted, and hence the orf288 gene is not expressed and no male sterility occurs. Accordingly, in the line, pollen normally grows, but the gene of the mitochondrial genome is transported only through a seed parent (maternal gene). Accordingly, another cytoplasmic male sterile line can be produced by crossing the line and a cytoplasmic male sterile line. Accordingly, a maintainer line plant can be produced by the method.

With regard to a crop whose genomic information is unknown, a cytoplasmic male sterile line and a maintainer line may be produced by, for example, the following method:
(1) the complete genome sequences of a nucleus and mitochondria are determined;
(2) an orf288 homologous gene is identified from the complete genome sequence of the mitochondria;
(3) the orf288 homologous gene is knocked out with mito-TALENs, and a null segregant from which the orf288 homologous gene has been deleted and out of which T-DNA, on which the mito-TALENs board, has fallen is selected;
(4) Rf-like PPR genes are sampled from the complete genome sequence of the nucleus, and an Rf-like PPR gene satisfying the following conditions is selected: the gene is expressed in a meiotic-stage anther; the gene has a mitochondrial targeting signal sequence; and the gene encodes 10 or more, more preferably 15 or more PPR motifs;
(5) a CRISPR-Cas9 binary vector for knocking out the selected Rf-like PPR gene is constructed, and is genetically introduced into an original variety and an orf288 homologous gene-deleted line; and
(6) a line obtained by knocking out the Rf-like PPR gene in the original variety expresses the orf288 homologous gene to become a cytoplasmic male sterile line, and a line obtained by knocking out the Rf-like PPR gene in the orf288 homologous gene-deleted line may be utilized as a maintainer line.

### Plant

The present invention also provides a cytoplasmic male sterile line plant obtained by knocking out, in a restorer line plant, which is a plant having an orf288 gene in its mitochondrial genome and having a pentatricopeptide repeat protein (PPR) gene in its nuclear genome, the PPR gene in the nuclear genome.

In addition, the present invention also provides a maintainer line plant, which is obtained by knocking out, in a restorer line plant, which is a plant having an orf288 gene in its mitochondrial genome and having a pentatricopeptide repeat protein (PPR) gene in its nuclear genome, the orf288 gene in the mitochondrial genome and the PPR gene in the nuclear genome.

Further, the present invention also provides a combination of plants of three lines including: a restorer line plant, which is a plant having an orf288 gene in its mitochondrial genome and having a pentatricopeptide repeat protein (PPR) gene in its nuclear genome; a cytoplasmic male sterile line plant obtained by knocking out, in the restorer line plant, the PPR gene in the nuclear genome; and a maintainer line plant obtained by knocking out, in the restorer line plant, the PPR gene in the nuclear genome and the orf288 gene in the mitochondrial genome. The combination is useful because an F₁ hybrid plant can be produced by crossing the plants of the three lines with plants of three lines of other varieties. Those plants of the respective lines may be produced by the above-mentioned methods.

In addition, the present invention also provides an F₁ hybrid plant, which is produced through use of the above-mentioned combination of plants of three lines and a combination of plants of three lines of other varieties by a three-line method. In the embodiment, an F₁ hybrid plant can be produced by using a known technique, that is, the three-line method except that the above-mentioned combination of the present invention formed of the cytoplasmic male sterile line plant, the maintainer line plant, and the restorer line plant is used.

Specific embodiments of the present invention are described in detail below by way of Examples. However, the present invention is not limited to the following Examples.

### EXAMPLES

### Reference Example 1

Proof that Standard Variety “Nipponbare” of Japonica Rice Carries Potential CMS-Causing Gene

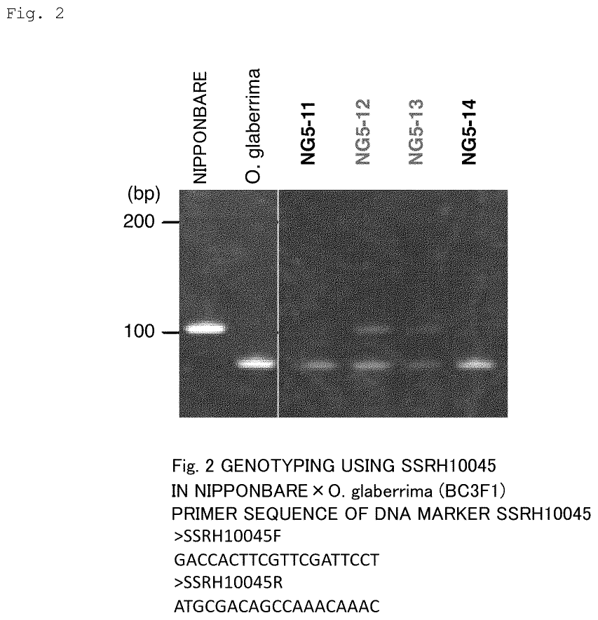

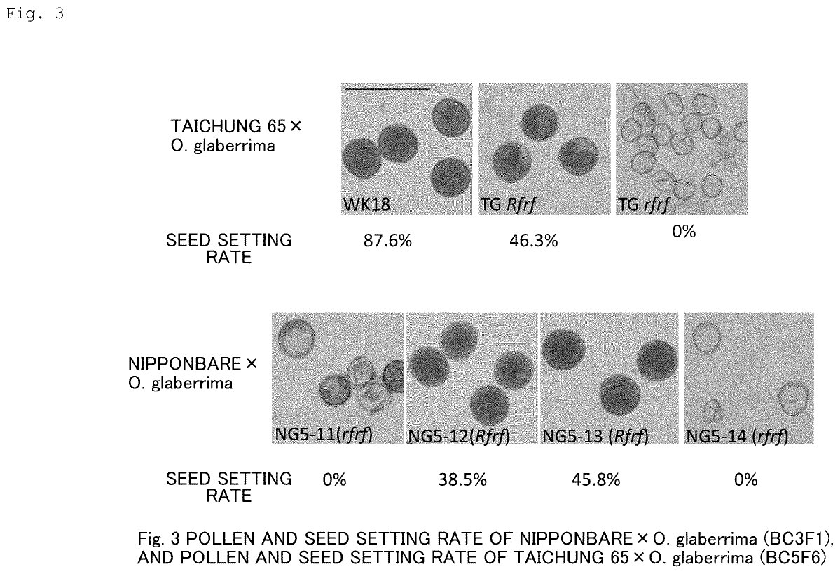

A BC3F1 plant was produced by successively backcrossing japonica rice “Nipponbare” with an O. glaberrima variety IRGC103777. At that time, marker selection was performed by using a DNA marker SSRH10045 capable of detecting a region on which a restorer-of-fertility gene boarded, and an individual in which the region on which the restorer-of-fertility gene boarded was heterozygous Nipponbare/O. glaberrima and an individual in which the region was homozygous O. glaberrima were selected (FIG. 2). As a result, the individual in which the region on which the restorer-of-fertility gene boarded was heterozygous set a seed because its pollen was normal, but the individual in which the region was homozygous O. glaberrima did not set any seed because its pollen was distorted and hence no starch accumulated therein (FIG. 3).

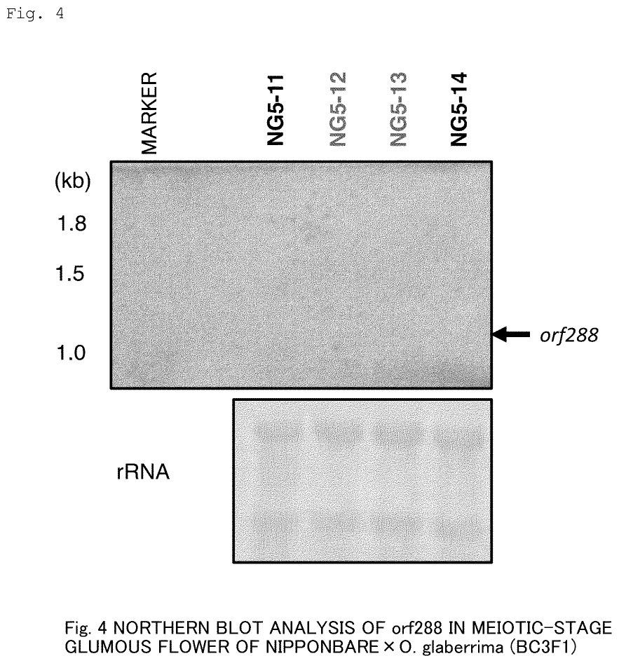

In addition, when the expression of orf288 was investigated by northern blot analysis through use of a meiotic-stage anther, substantially no orf288 RNA was able to be detected in the individual in which the region on which the restorer-of-fertility gene boarded was heterozygous, but orf288 RNA was able to be detected in the individual in which the region was homozygous O. glaberrima (FIG. 4).

Thus, it was concluded that the orf288 gene was a candidate for the potential CMS-causing gene of the standard variety “Nipponbare” of the japonica rice.

### Reference Example 2

### Narrow-Down of Restorer-Of-Fertility Gene Candidates

It was predicted that the RNA of the orf288 was subjected to processing by a restorer-of-fertility gene, and hence the restorer-of-fertility gene was a PPR gene serving as a sequence-specific RNA-binding protein. Rf-like PPR genes, which are present in a restorer-of-fertility gene-boarded region reported in Non-patent Literature 8 on the basis of the genome sequence information of the “Nipponbare” and are expressed in an anther, are the following three genes, and the genes are restorer-of-fertility gene candidates: a gene whose locus ID in the RAP-DB (https://rapdb.dna.affrc.go.jp) is Os10g0495100 (named PPR461 because the gene encodes 461 amino acid sequences); a gene whose locus ID is Os10g0495200 (named PPR782 because the gene encodes 782 amino acid sequences); and a gene whose locus ID is Os10g0497300 (named PPR794 because the gene encodes 794 amino acid sequences). It has been known that one PPR motif of a PPR protein recognizes one nucleotide. When the number of the PPR motifs of each of the genes was predicted with TPRpred (https://toolkit.tuebingen.mpg.de/tools/tprpred), it was predicted that the PPR461 had 12 PPR motifs, and the PPR782 and the PPR794 each had 21 PPR motifs. Thus, the PPR461, the PPR782, or the PPR794 is expected to be a restorer-of-fertility gene.

### Example 1

### Demonstration Experiment of Production of Cytoplasmic Male Sterile Line (CMS Line)

The binary vector of a CRISPR-Cas9 system capable of knocking out one each, simultaneously two, or simultaneously three, of the genes Os10g0495100 (PPR461), Os10g0495200 (PPR782), and Os10g0497300 (PPR794) was constructed.

### Target Sequence for Knocking Out One Each of Genes (the Underlined Part is a PAM Sequence)

PPR461:
SEQ ID NO: 17
253 TACACCTACAGCGTTCTCATCGG 275
PPR782:
SEQ ID NO: 18
608 TCGTATAACACTGTCCTCAATGG 630
PPR794:
SEQ ID NO: 19
292 ATCGGTTCCTGCTGCTGCGCGGG 314

### Target Sequence for Knocking Out Three Genes Designed to be a Sequence Common to the Three Genes (the Underlined Part is a PAM Sequence)

SEQ ID NO: 20
TTATGCAAGGCTCAAGCTATGG

The above-mentioned target sequences were inserted into a Cas9, gRNA-integrated binary vector pZH_OsU6gRNA_MMCas9 (Non-patent Literature 6). To simultaneously disrupt two genes, two gRNA expression cassettes targeting the respective genes were connected in series, and were inserted into the binary vector.

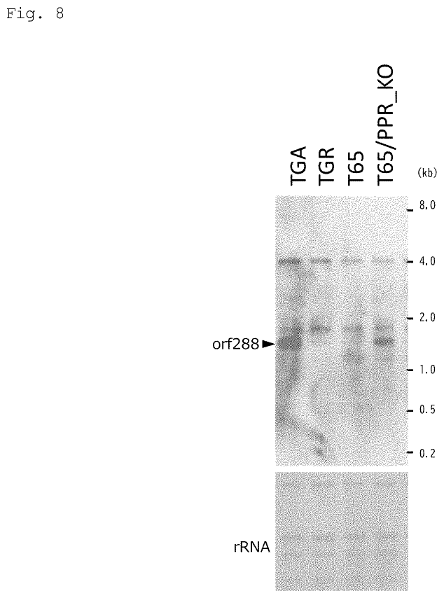

The genes were introduced into Taichung 65 by an Agrobacterium method through use of the above-mentioned vector. In the northern blot analysis of RNA extracted from a leaf of a null segregant individual obtained by introducing homozygous mutation into the PPR782 (from which four bases had been deleted), the PPR794 (from which five bases had been deleted), or the PPR461 (from which one base had been deleted) through use of the target sequence for knocking out three genes designed to be a sequence common to the three genes (SEQ ID NO: 20), the expression of the orf288 was detected (FIG. 8). Thus, the inventors have succeeded in releasing the suppression of the expression of the orf288 gene present in a mitochondrial genome by knocking out the PPR gene in a nuclear genome. In consideration of the results of Example 3 to be described later, an individual obtained by knocking out the PPR gene in nuclear genomic DNA can be expected to show male sterility.

### Example 2

### Demonstration Experiment of Production of Maintainer Line

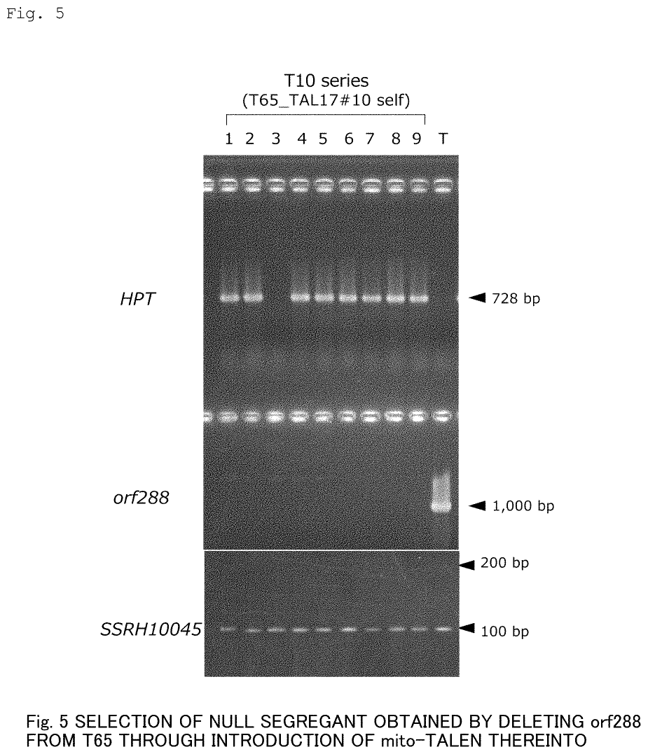

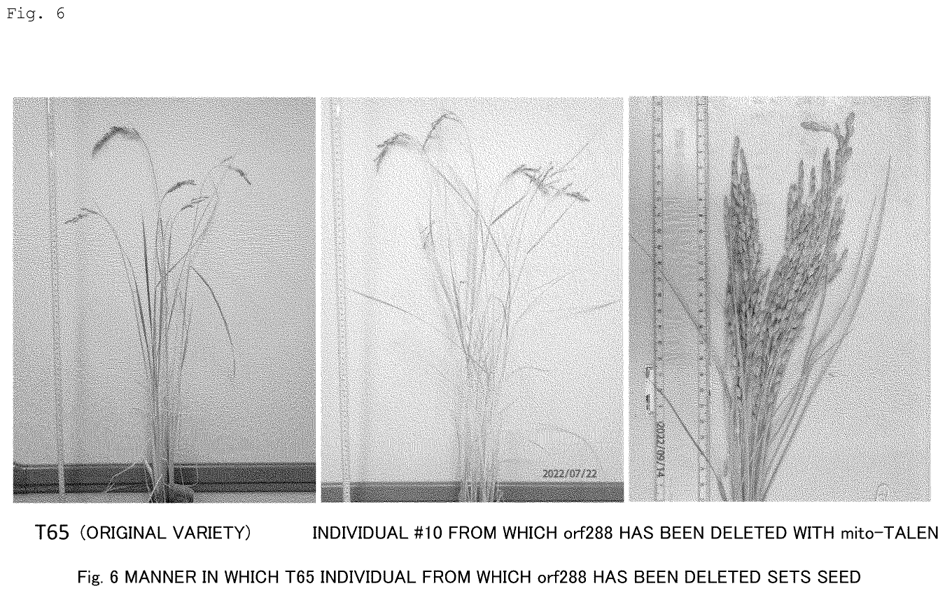

To disrupt the CMS-causing gene candidate orf288 of mitochondria through use of a genome editing technology mito-TALEN, TAL17 (DDBJ accession no. LC662754), which was one kind of mito-TALEN targeting the following sequences, was genetically introduced into Taichung 65 to provide two regenerated individuals from each of which the orf288 had been deleted (line names #9 and #10). In a T2 plant, a null segregant out of which T-DNA (HPT) had fallen was selected (FIG. 5), and was inbred so that its seeds were proliferated. No growth abnormality was observed in the plant from which the orf288 gene had been deleted, and the seed setting rates of the regenerated next generations were as follows: the seed setting rate of the line #9 was 95%, and that of the line #10 was 98% (FIG. 6). It was revealed from the foregoing that the orf288 gene was not indispensable to the growth of rice. Further, the above-mentioned Cas9, gRNA-integrated binary vector for knocking out the PPR461, the PPR782, and the PPR794 was introduced into a null segregant from which the orf288 gene had been deleted. The individual in which the orf288, the PPR461, the PPR782, and the PPR794 have been knocked does not show any male sterility. The null segregant is selected and adopted as a maintainer line.

Primer Sequence of PCR for Detecting orf288

>orf288_F314
SEQ ID NO: 21
TACGAACAACCGAGGATGAG
>orf288_R314
SEQ ID NO: 22
GATTCCCCCTTCTCTCAGCC

### Primer Sequence for Detecting Hygromycin-Resistant Gene for Selecting Null Segregant

>HPT-ZF
SEQ ID NO: 23
GAGAGCCTGACCTATTGCAT
>HPT-ZR
SEQ ID NO: 24
TCGGCGAGTACTICTACACA

### Example 3

In genome-edited individuals obtained by genetically disrupting nucleus-controlling gene candidates (the PPR461, the PPR782, and the PPR794) in an orf288-deleted line and an original variety carrying the orf288 with CRISPR-Cas9, the individual of the orf288-deleted line does not become male sterile, and the individual of the original variety carrying the orf288 becomes male sterile. The CMS line produced in the section “Demonstration Experiment of Production of Cytoplasmic Male Sterile Line (CMS Line)” is crossed with the maintainer line produced in the section “Demonstration Experiment of Production of Maintainer Line.” A next-generation individual shows male sterility. When the CMS line produced in the section “Demonstration Experiment of Production of Cytoplasmic Male Sterile Line (CMS Line)” is crossed with the original variety, the resultant F₁ plant restores its fertility. Japonica rice varieties are substantially identical in maternal origin to each other, and hence it is conceivable that all the varieties each carry the orf288 and each carry, in its nucleus, a restorer-of-fertility gene for suppressing its expression.

### Reference Example 3

### Application to Crop Except Rice

orf288 Homologous Gene Present in Mitochondrial Genome

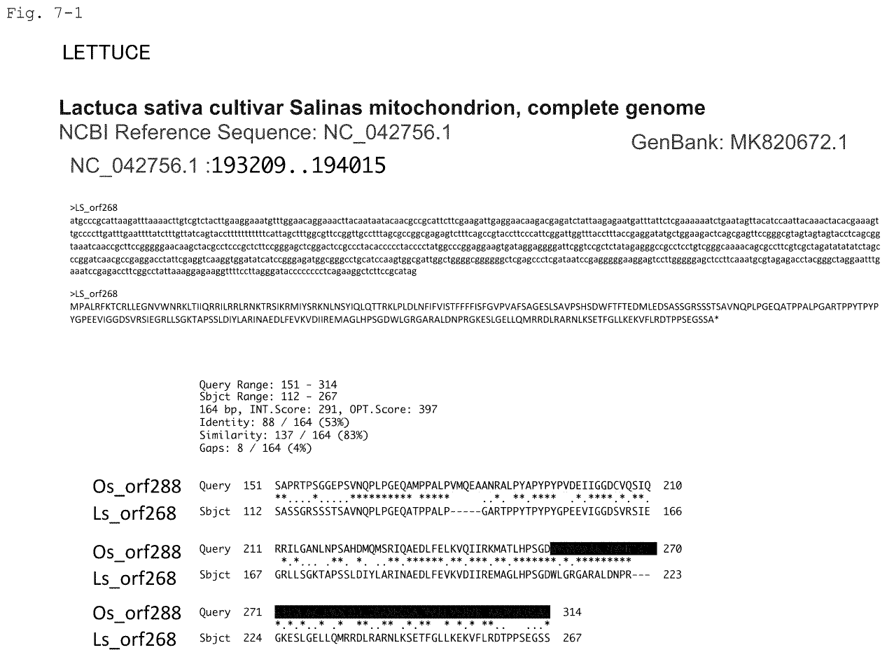

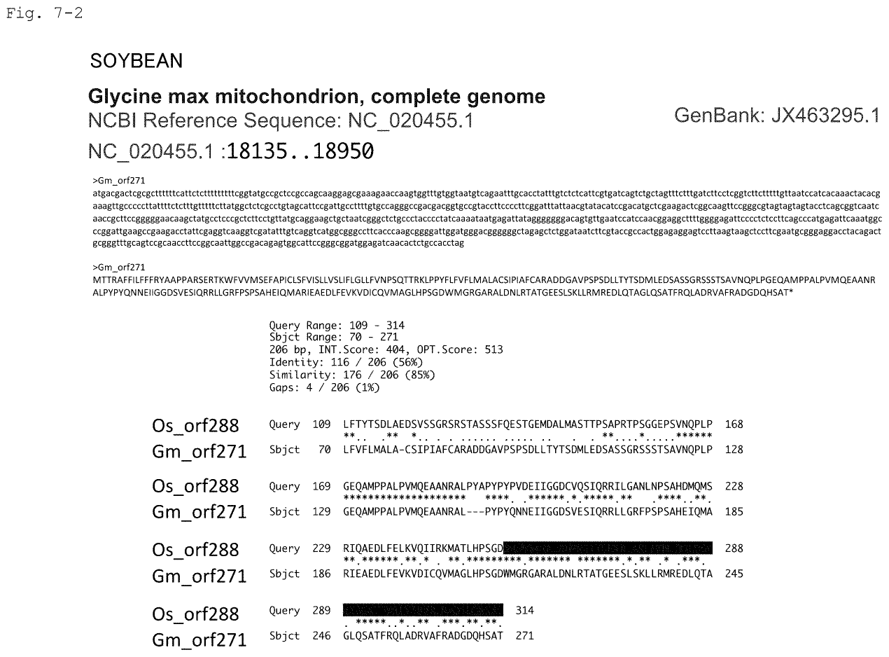

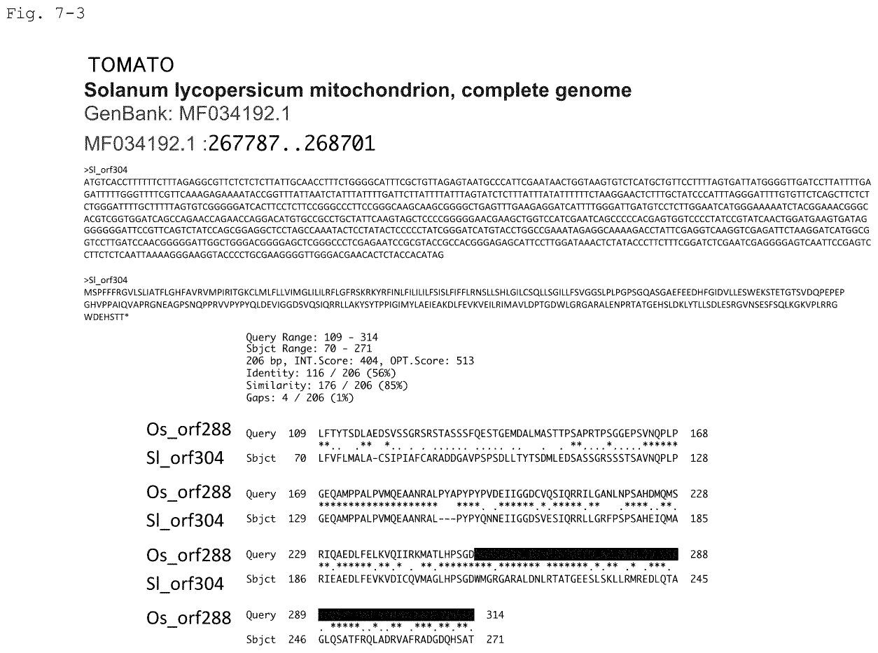

It has been reported that an ORF288 protein binds to the subunit COX11 of a cytochrome C oxidase complex as in a CW-type CMS-causative factor WA352 (Non-patent Literature 4). Accordingly, it is predicted that when the activity of the cytochrome C oxidase complex is inhibited and reactive oxygen species are produced, male sterility is caused. An amino acid sequence in a COX11 interaction region has been reported (FIG. 7).

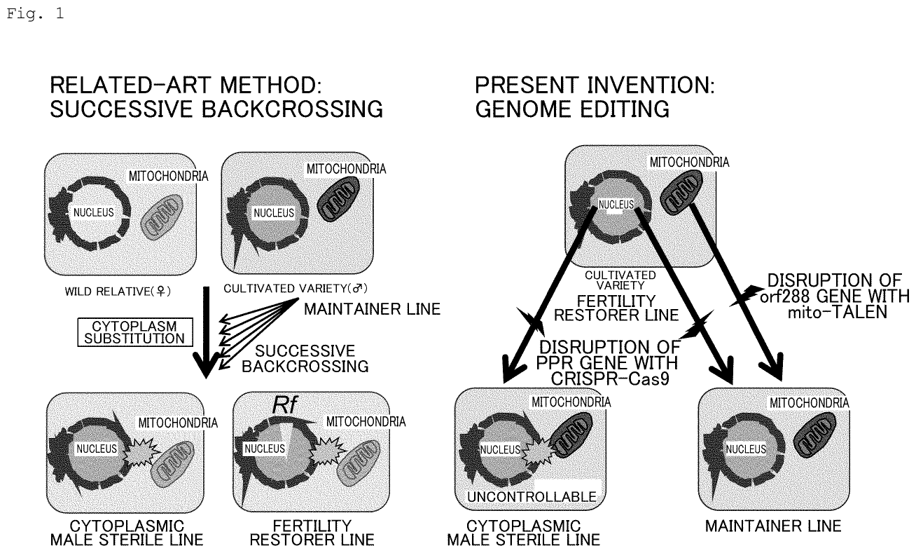

When an NCBI-BLASTP search is performed by using the amino acid sequence of the ORF288 as a query, a homologous gene product can be detected in, for example, a crop shown in Table 1. Examples of a lettuce, a soybean, and a tomato are illustrated as examples of homology between amino acid sequences (the amino acid sequence of a lettuce orf268: SEQ ID NO: 14, the amino acid sequence of a soybean orf271: SEQ ID NO: 15, and the amino acid sequence of a tomato orf304: SEQ ID NO: 16) in FIG. 7-1 to FIG. 7-3. The base sequence of the lettuce orf268 is as set forth in SEQ ID NO: 11. The base sequence of the soybean orf271 is as set forth in SEQ ID NO: 12. In addition, the base sequence of the tomato orf304 is as set forth in SEQ ID NO: 13. It can be predicted that those ORF288 homologous genes are potential CMS-causing genes because amino acid sequences in the COX11 interaction region (sequences illustrated with blue and green markers) show high homology.

TABLE 1
ORF288 Homologous gene

| Crop | | GenBank registration number |
|---|---|---|
| name | Scientific name | of mitochondrial genome |
| Lettuce | Lactuca sativa | MK820672 |
| Soybean | Glycine max | JX463295 |
| Tomato | Solanum lycopersicum | MF034192 |

### Reference Example 4

### Search in Database of PPR Gene Involved in Fertility Restoration Present in Nucleus

In addition, a plurality of Rf-like PPR genes have been identified by selecting a gene having an E-value of 1e-100 or less through an NCBI-BLASTP search using the sequence of a restorer-of-fertility gene PPR791 (GenBank accession no. AB110016) for the BT-type CMS of rice, a restorer-of-fertility gene PPR-B (GenBank accession no. AJ550021) for an Ogura-type CMS rapeseed, or a restorer-of-fertility gene PPR592 (GenBank accession no. AY102719) for petunia CMS as a query (Non-patent Literature 7). It has been reported that PPR proteins encoded by the Rf-like PPR genes each basically carry 15 to 20 PPR motifs.

When an NCBI-BLASTP search is performed on japonica rice by the same approach through use of the amino acid sequence of the restorer-of-fertility gene PPR791 (GenBank accession no. AB110016) for the BT-type CMS of rice as a query, proteins each having an E-value of 1e-100 or less are selected, and proteins predicted from the genome annotation pipeline of an NCBI Reference Sequence database are sampled, such proteins as shown in Table 2 are sampled. The restorer-of-fertility gene functions in mitochondria, and hence when proteins each having a mitochondrial targeting signal sequence are selected by using a program Predotar (https://urgi.versailles.inra.fr/predotar/) for predicting the intracellular localization of a protein, 12 proteins are selected. Next, when the number of PPR motifs is predicted by using a prediction program TPRpred (https://toolkit.tuebingen.mpg.de/tools/tprpred), a PPR protein having 15 or more PPR motifs can be selected. The following three genes can be sampled as genes each satisfying all of the foregoing conditions: the Os10g0497300, an Os08g0107700, and an Os08g0110200. Further, when the degree of expression displayed on a rice database RAP-DB (https://rapdb.dna.affrc.go.jp) is investigated in TENOR based on mRNA-seq data, and a gene expressed in a panicle before panicle emergence is selected under such a condition that the degree of expression satisfies TPM>1, one gene, that is, the Os10g0497300 can be selected. The Os10g0497300 is the PPR794 revealed in Example 1, and hence a restorer-of-fertility gene candidate that controls orf288 RNA can be selected on the database.

TABLE 2
PPR791 homologous gene in japonica rice

| | | | | Expression |
|---|---|---|---|---|
| Protein | | Prediction of | Number | in panicle |
| having high | | intracellular | of PPR | before panicle |
| homology in | Locus ID of | localization | motifs | emergence |
| NCBI-BLASTP | RAP-DB | (Predotar) | (TPRpred) | (TENOR) |
| XP_015651079.1 | 0s08g0248400 | none | 21 | 0.5 |
| XP_015614273.1 | 0s10g0497300 | mitochondrial | 17 | 2.6 |
| XP_025883348.1 | 0s08g0107700 | mitochondrial | 21 | 0.4 |
| XP_015614274.1 | 0s10g0497300 | mitochondrial | 13 | 2.6 |
| XP_015650385.1 | 0s08g0110200 | possibly | 18 | 1 |
| | | mitochondrial | | |
| XP_015613590.1 | 0s10g0495400 | mitochondrial | 12 | 6 |
| XP_025876776.1 | 0s10g0495100 | possibly | 12 | 1.2 |
| | | mitochondrial | | |
| XP_015614861.1 | 0s10g0499500 | mitochondrial | 13 | 0.5 |
| XP_015614275.1 | 0s10g0497366 | mitochondrial | 9 | 2 |
| XP_015613592.1 | 0s10g0495400 | mitochondrial | 9 | 6 |
| XP_015613591.1 | 0s10g0495400 | mitochondrial | 10 | 6 |
| XP_025876777.1 | 0s10g0495400 | mitochondrial | 10 | 6 |
| XP_015635653.2 | 0s04g0350000 | none | 20 | 1.3 |
| XP_025880832.1 | 0s04g0351300 | none | 17 | 0 |
| XP_015635659.2 | 0s04g0350000 | none | 18 | 1.3 |
| XP_015614283.1 | 0s10g0497432 | mitochondrial | 6 | 7.6 |

Similarly, when an NCBI-BLASTP search is performed on plant species, which are limited to a lettuce, a soybean, and a tomato, through use of the amino acid sequence of the restorer-of-fertility gene PPR-B (GenBank accession no. AJ550021) for an Ogura-type CMS rapeseed or the restorer-of-fertility gene PPR592 (GenBank accession no. AY102719) for petunia CMS as a query, genes each having an E-value of 1e-100 or less are selected, and RE-like PPRs are selected under such conditions that each of the PPRs has a mitochondrial targeting signal sequence and encodes 15 or more PPR motifs, genes encoding proteins shown in Table 3 are sampled. Base sequence information can be acquired from the protein IDP of Table 3 by using an NCBI database. When RNA-seq data or the like is available, as described in Example 1, only genes that are expressed in inflorescence before blooming (if possible, meiotic-stage anthers) can be further narrowed down and adopted as restorer-of-fertility gene candidates.

TABLE 3
RF-like PPR protein having mitochondrial
targeting signal sequence

| | Protein ID | Number of PPR motifs |
|---|---|---|
| Lettuce | XP_023750012.1 | 16 |
| | XP_023735348.1 | 16 |
| | XP_023770534.1 | 15 |
| | XP_023758522.1 | 15 |
| | XP_023768240.1 | 17 |
| Soybean | XP_014629135.1 | 15 |
| | XP_006585147.1 | 15 |
| | XP_003520004.3 | 15 |
| | XP_014629134.1 | 15 |
| | XP_025984262.1 | 16 |
| | XP_006599382.1 | 15 |
| | XP_006599487.1 | 15 |
| | XP_003533262.1 | 15 |
| | XP_003518493.2 | 16 |
| | XP_014624504.1 | 15 |
| | XP_040862178.1 | 15 |
| Tomato | XP_004238596.1 | 15 |
| | XP_004240414.1 | 15 |
| | XP_025885961.1 | 22 |
| | XP_025884302.1 | 17 |
| | XP_004237581.1 | 17 |
| | XP_010323884.1 | 21 |

When an NCBI-BLASTP search is performed by the same approach, and at that time, searching objects are limited to crops of interest, the Rf-like PPR genes of the crops can be identified. It is assumed that one of the Rf-like PPR genes is involved in the RNA control of an orf288 homologous gene. Accordingly, it is expected that a cytoplasmic male sterile line can be produced by knocking out those Rf-like PPR genes with CRISPR-Cas9.

1. A method of producing a cytoplasmic male sterile line plant, comprising a step of knocking out, in a plant having an orf288 gene in mitochondrial genomic DNA thereof and having a pentatricopeptide repeat protein (PPR) gene in nuclear genomic DNA thereof, the PPR gene in the nuclear genomic DNA.

2. A method of producing a maintainer line plant, comprising the steps of:
knocking out, in a plant having an orf288 gene in mitochondrial genomic DNA thereof and having a PPR gene in nuclear genomic DNA thereof, the PPR gene in the nuclear genomic DNA; and
knocking out the orf288 gene in the mitochondrial genomic DNA.

3. The method according to claim 1, wherein the PPR gene satisfies all of the following requirements:
(1) the PPR gene is expressed in an anther of the plant;
(2) the PPR gene has a mitochondrial targeting signal; and
(3) the PPR gene has 10 or more PPR motifs.

4. The method according to claim 2, wherein the orf288 gene includes a base sequence encoding an amino acid sequence as set forth in SEQ ID NO: 3 or a base sequence encoding an amino acid sequence having a similarity of 80% or more to the amino acid sequence as set forth in SEQ ID NO: 3 in blast analysis.

5. A combination of plants of three lines, comprising:
a restorer line plant, which is a plant having an orf288 gene in mitochondrial genomic DNA thereof and having a pentatricopeptide repeat protein (PPR) gene in nuclear genomic DNA thereof;
a cytoplasmic male sterile line plant obtained by knocking out, in the restorer line plant, the PPR gene in the nuclear genomic DNA; and
a maintainer line plant obtained by knocking out, in the restorer line plant, the PPR gene in the nuclear genomic DNA and the orf288 gene in the mitochondrial genomic DNA.

6. An F₁ hybrid plant, which is produced through use of the combination of plants of three lines of claim 5 and a combination of plants of three lines of other varieties by a three-line method.

7. The combination according to claim 5, wherein the PPR gene satisfies all of the following requirements:
(1) the PPR gene is expressed in an anther of the plant;
(2) the PPR gene has a mitochondrial targeting signal; and
(3) the PPR gene has 10 or more PPR motifs.

8. The combination according to claim 5, wherein the orf288 gene includes a base sequence encoding an amino acid sequence as set forth in SEQ ID NO: 3 or a base sequence encoding an amino acid sequence having a similarity of 80% or more to the amino acid sequence as set forth in SEQ ID NO: 3 in blast analysis.

9. The F₁ hybrid plant according to claim 6, wherein the PPR gene satisfies all of the following requirements:
(1) the PPR gene is expressed in an anther of the plant;
(2) the PPR gene has a mitochondrial targeting signal; and
(3) the PPR gene has 10 or more PPR motifs.

10. The F₁ hybrid plant according to claim 6, wherein the orf288 gene includes a base sequence encoding an amino acid sequence as set forth in SEQ ID NO: 3 or a base sequence encoding an amino acid sequence having a similarity of 80% or more to the amino acid sequence as set forth in SEQ ID NO: 3 in blast analysis.
